(19) United States
(12) Patent Application Publication
(10) Pub. No.: US 2024/0066243 A1
(43) Pub. Date: Feb. 29, 2024

(54) HIGH FLOW RESPIRATORY THERAPY DEVICE AND METHOD THROUGH BREATH SYNCHRONIZATION

(71) Applicant: MEK CO., LTD., Paju-si, Gyeonggi-do (KR)

(72) Inventor: Jong Cheol KIM, Goyang-si, Gyeonggi-do (KR)

(21) Appl. No.: 18/271,929

(22) PCT Filed: Apr. 15, 2021
(86) PCT No.: PCT/KR2021/004770

§ 371 Date: Jul. 12, 2023

(30) Foreign Application Priority Data
Jan. 12, 2021 (KR) 10-2021-0004258

(51) Int. Cl. A61M 16/00; A61M 16/10

(52) U.S. Cl. CPC A61M 16/022; A61M 16/0003; A61M 16/0066; A61M 16/1005; A61M 16/1075

(57) ABSTRACT

A high flow respiratory therapy device includes: a breathing pattern monitoring part for monitoring a flow rate change and a pressure change of a mixed gas supplied to a patient so as to collect breathing pattern information of the patient; an inspiratory effort point detection part for detecting an inspiratory effort point, at which the patient tries to start inhalation, from the breathing pattern information of the patient; an expiratory effort point detection part for detecting an expiratory effort point, at which the patient tries to start exhalation, from the breathing pattern information of the patient; and a supply flow rate control part for increasing the flow rate of the supplied mixed gas when the breath of the patient reaches the inspiratory effort point, and for reducing the flow rate of the supplied mixed gas when the breath of the patient reaches the expiratory effort point.

### TECHNICAL FIELD

The present invention relates to a high flow respiratory therapy device, and more specifically, to high flow respiratory therapy device and method through breath synchronization, which can detect the time point when a patient attempts to start inhalation and the time point when a patient attempts to start exhalation, reduce a dead space by supplying a high flow of mixed Gas during inhalation, and reduce the patient's respiratory effort by decreasing a flow rate of mixed gas during exhalation.

### BACKGROUND ART

High flow respiratory therapy means therapy assisting a patient's respiration by relatively reducing a dead space where gas exchange does not occur by delivering air heated and humidified at concentration higher than the oxygen concentration in the atmosphere, at a flow rate two to three times or more than the patient's breathing volume.

In general, most of the air goes into the alveoli for gas exchange, and some remains in the respiratory tract (or airway). The volume of air that stays in the airway is called the dead space, where no gas exchange takes place during actual respiration.

For instance, in a case in which the volume of the dead space is about 150 ml when the tidal volume of a healthy person is 500 ml, the actual tidal volume becomes 350 ml. However, a patient with decreased respiratory capacity, whose tidal volume is only 300 ml, would have an actual breathing volume of 150 ml after excluding the dead space of 150 ml, which is less than half the normal level. Therefore, if the patient's tidal volume is not sufficiently larger than the dead space, the patient may experience respiratory failure, namely, respiratory-deficient.

Therefore, the patient may breathe faster or consume a lot of energy to maintain normal O2 saturation (SaO2 or SpO2), and it may deteriorate the patient's condition.

To solve the above problem, conventionally, a method of providing a constant flow of mixed gas to a patient using a high flow respiratory therapy device have been used. The administration method may include using a nasal cannula, which is a thin tube inserted into both nostrils and hung over the ears, or an oxygen mask.

However, the conventional high flow respiratory therapy device can reduce the patient's effort to breathe during inhalation because a supply flow to the patient is greater than an inhalation flow by the patient's spontaneous respiration. However, the conventional high flow respiratory therapy device may give burden to the patient's expiratory effort since air resistance increases compared to the spontaneous respiration due to high flow nasal delivery during exhalation.

Additionally, the start of the patient's inhalation entirely depends on the patient's will to breathe. In contrast, the conventional high flow respiratory therapy device has an effect of the high flow therapy since delivering mixed gas at the same flow rate regardless of inhalation and exhalation time points, which may vary according to the patient's condition, but cannot reduce the burden on the patient's inspiratory effort or expiratory effort.

Especially, the high flow respiratory therapy using a nasal cannula diversely detects resistance, volume, and breathing effort according to the size of the patient's nostrils, the state of cannula mounting, and the anatomical characteristics of the nasal cavity and upper airway even though mixed gas of the same flow rate is supplied, so it is difficult to perform active respiratory therapy to reduce the expiratory effort due to technical difficulty in synchronized detection (detection at the time of inhalation/exhalation).

### DISCLOSURE

### Technical Problem

Accordingly, the present invention has been made in view of the above-mentioned problems occurring in the related art, and it is an object of the present invention to provide high flow respiratory therapy device and method through breath synchronization, which can detect the time point when a patient attempts to start inhalation and the time point when a patient attempts to start exhalation, reduce a dead space by supplying a high flow of mixed gas during inhalation, and reduce the patient's respiratory effort by decreasing a flow rate of mixed gas during exhalation.

It is another object of the present invention to provide high flow respiratory therapy device and method through breath synchronization, which detects only the time point when a patient tries to start exhalation to usually supply mixed gas of a basic flow rate and to supply mixed gas of an minus auxiliary exhalation flow rate which is lower than the basic flow rate when exhalation starts after completion of the patient's inhalation, thereby reducing the patient's expiratory effort, and returning to the basic flow rate by increasing the flow rate in proportion to the reduction of the patient's expiratory effort.

### Technical Solution

To accomplish the above-mentioned objects, according to the present invention, there is provided a high flow respiratory therapy device through breath synchronization including: a breathing pattern monitoring part, which monitors flow rate and pressure changes of a mixed gas supplied to a patient and collects the patient's breathing pattern information; an inspiratory effort point detection part, which detects an inspiratory effort point, at which the patient tries to start inhalation, from the patient's breathing pattern information; an expiratory effort point detection part, which detects a expiratory effort point, at which the patient tries to start exhalation, from the patient's breathing pattern information; and a supply flow control part, which increases the flow of the mixed gas when the patient's respiration becomes the inspiratory effort point and decreases the flow of the mixed gas when the patient's respiration becomes the expiratory effort point.

Moreover, the high flow respiratory therapy device further includes a breathing synchronization unit, which includes a breathing pattern monitoring part, an inspiratory effort point detection part, and an expiratory effort point detection part, and synchronizes the extracted inspiratory effort point and the expiratory effort point with the patient's breathing pattern information, wherein the supply flow control part synchronizes the flow control of the mixed gas with respect to the inspiratory effort point and expiratory effort point synchronized in the breathing synchronization unit.

In another aspect of the present invention, there is provided a high flow respiratory therapy device through breathing synchronization including: a breathing pattern monitoring part, which monitors one or more of flow and pressure changes of a mixed gas supplied to a patient and collects patient's breathing pattern information; an expiratory effort point detection part, which detects an expiratory effort point at which the patient tries to start exhalation from the collected breathing pattern information; and a supply flow control part, which supplies the mixed gas of a set basic flow when the patient's respiration starts, and reduces the flow rate of the mixed gas by an minus auxiliary (assist) exhalation flow rate (relief flow) at the expiratory effort point to reduce the patient's expiratory effort.

Additionally, the high flow respiratory therapy device further includes a breathing synchronization unit, which includes a breathing pattern monitoring part, and an expiratory effort point detection part and synchronizes the extracted expiratory effort point with the patient's breathing pattern information, wherein supply flow control part synchronizes the flow control of the mixed gas according to the expiratory effort point synchronized in the breathing synchronization unit.

In another aspect of the present invention, there is provided a therapy method in a high flow respiratory therapy device, including: a step in which a breathing pattern monitoring part monitors flow rate and pressure changes of a mixed gas supplied to a patient and collects the patient's breathing pattern information; a step in which a detection part of a control part detects an inspiratory effort point, at which the patient tries to start inhalation, and an expiratory effort point, at which the patient tries to start exhalation, from the patient's breathing pattern information, or detects only the expiratory effort point; and a step in which a supply flow control part of the control part actively controls the flow rate of the mixed gas in response to the inspiratory effort point or the expiratory effort point.

In addition, the therapy method further includes, after the step of detecting, a step in which the breathing synchronization part of the control part synchronizes the detected inspiratory effort point or the expiratory effort point with the patient's breathing pattern information, wherein in the step of controlling the flow of the mixed gas, the supply flow control part of the control part synchronizes the flow control of the mixed gas in response to the inspiratory effort point and expiratory effort point synchronized in the breathing synchronization part.

### Advantageous Effects

The present invention supplies a high flow of a mixed gas sufficient to ventilate the patient's nasal cavity with fresh air during inhalation, thereby clinically reducing the dead space, and reducing the patient's respiratory effort by reducing the patient's expiratory effort.

### DESCRIPTION OF DRAWINGS

FIGS. 1 and 2 are diagrams illustrating the configuration of a high flow respiratory therapy device according to an embodiment of the present invention.

FIG. 3 is a diagram illustrating the configuration of the high flow respiratory therapy device according to a first embodiment of the present invention in the device of FIGS. 1 and 2.

FIG. 4 is a graph showing a value that changes in flow rate of a mixed gas are monitored according to the first embodiment of the present invention.

FIGS. 5 and 6 are waveform diagrams illustrating an example of extracting an inspiratory effort point and an expiratory effort point of a patient according to the patient's respiratory changes according to the first embodiment of the present invention.

FIG. 7 is an operation flow chart for depicting a high flow respiratory therapy method through breathing synchronization according to the first embodiment of the present invention.

FIG. 8 is a diagram illustrating the configuration of a high flow respiratory therapy device according to a second embodiment of the present invention.

FIG. 9 is a graph showing a value that changes in flow rate of a mixed gas are monitored according to the second embodiment of the present invention.

FIG. 10 is a waveform diagram illustrating an example of extracting an inspiratory effort point and an expiratory effort point of a patient according to the patient's respiratory changes according to the second embodiment of the present invention.

FIG. 11 is an operation flow chart for depicting a high flow respiratory therapy method through breathing synchronization according to the second embodiment of the present invention.

### MODE FOR INVENTION

Hereinafter, embodiments of the present invention will be described in detail with reference to the accompanying drawings.

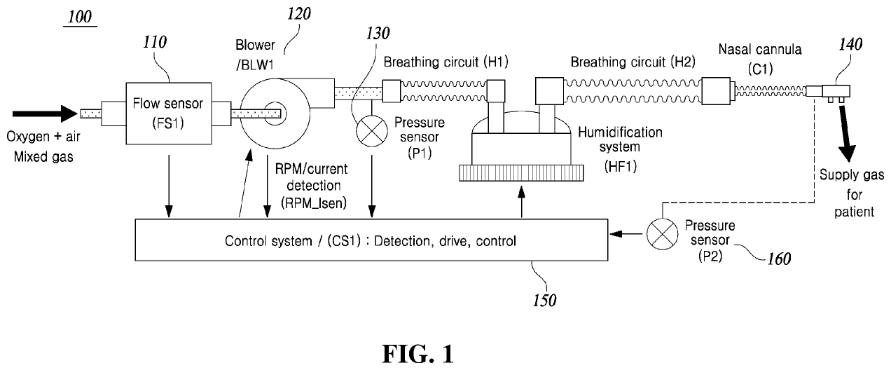

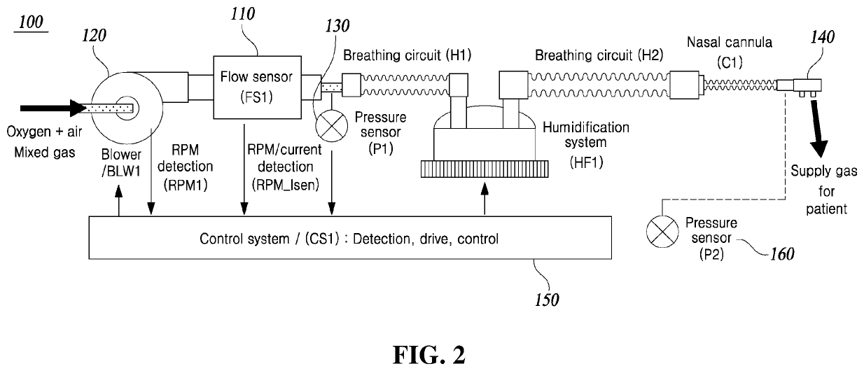

FIGS. 1 and 2 are diagrams illustrating the configuration of a high flow respiratory therapy device according to an embodiment of the present invention.

The high flow respiratory therapy device 100 according to an embodiment of the present invention includes a mixed gas generating unit, which generates mixed gas containing oxygen and air, a flow sensor 110 (in FIGS. 1 and 2), a blower 120 (in FIGS. 1 and 2), a pressure sensor 130 (in FIGS. 1 and 2), a humidifier HF1 (in FIGS. 1 and 2), a nasal cannula C1 (in FIGS. 1 and 2) which has a supply hose 140 (in FIGS. 1 and 2), and a control part 150 (in FIGS. 1 and 2).

The flow sensor FS1 may be installed at the front end (see FIG. 1) or the rear end (see FIG. 2) of the blower 120 depending on the position and configuration to be sensed.

The pressure sensor 130 may be additionally placed close to the patient like P2 of FIGS. 1 and 2. The pressure sensor 160 at the rear end can measure the flow rate and pressure supplied to the patient close to reality, but the measurement results may significantly vary depending on the mounted state of the nasal cannula 140 or the installation status of the cannula 140. By the above configuration, the high flow respiratory therapy device 100 administers a heated and humidified mixed gas with oxygen concentration higher than that of the atmosphere to the patient. In this instance, the high flow respiratory therapy device 100 adjusts the flow rate and flow volume of the mixed gas depending on the inhalation and exhalation points of the patient's breath.

In addition, in order to solve the problem that the resistance, volume, and respiratory effort are differently detected according to the patient's nostril size, the installation status of the cannula, and the anatomical characteristics of the nasal cavity and the upper airway, the high flow respiratory therapy device 100 according to an embodiment of the present invention detects the inhalation and exhalation points based on the pressure and the flow rate supplied to the patient and synchronizes them with the patient's respiratory cycle. For this, the control part 150 must precede the breathing synchronization process which monitors and synchronizes the patient's breath before treatment.

Therefore, the high flow respiratory therapy device 100 according to an embodiment of the present invention reduces the patient's respiratory effort by increasing or decreasing the flow rate of the mixed gas at each inhalation point or exhalation point, referring to the synchronized inhalation and exhalation points in the control part 150.

Specifically, two synchronization plans for the high flow respiratory therapy device 100 according to an embodiment of the present invention will be provided.

### First Embodiment

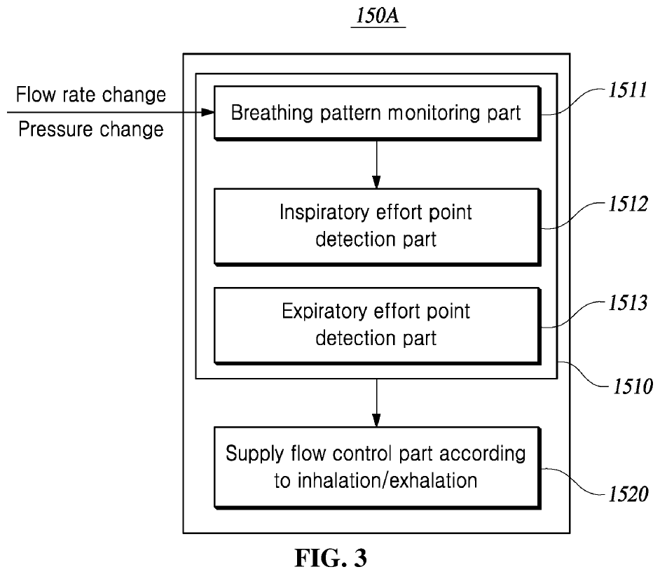

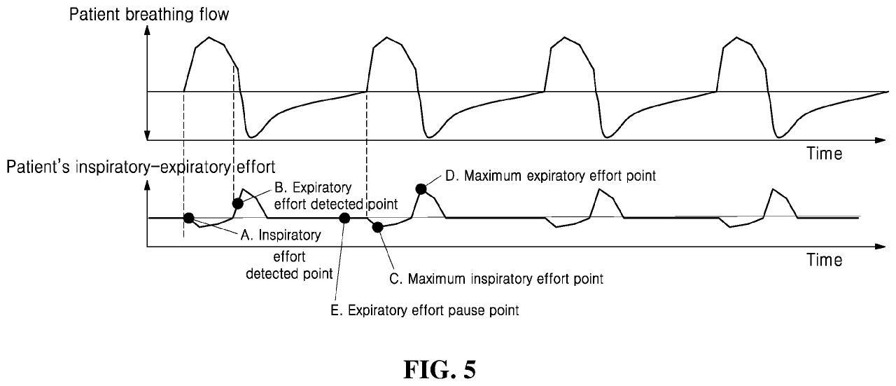

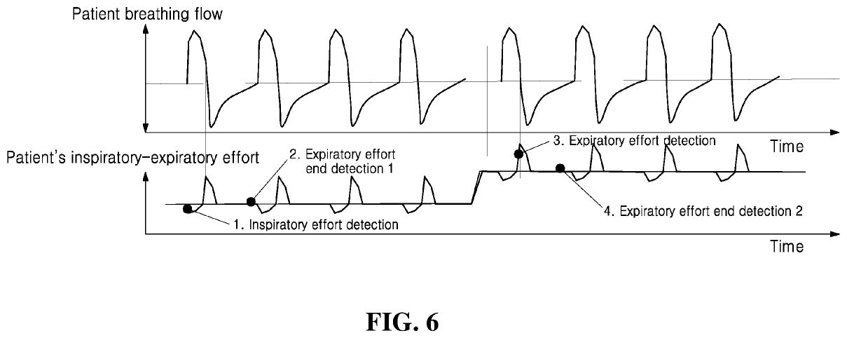

FIG. 3 is a diagram illustrating the configuration of the high flow respiratory therapy device according to a first embodiment of the present invention in the device of FIGS. 1 and 2, FIG. 4 is a graph showing a value that changes in flow rate of a mixed gas are monitored according to the first embodiment of the present invention, and FIGS. 5 and 6 are waveform diagrams illustrating an example of extracting an inspiratory effort point and an expiratory effort point of a patient according to the patient's respiratory changes according to the first embodiment of the present invention.

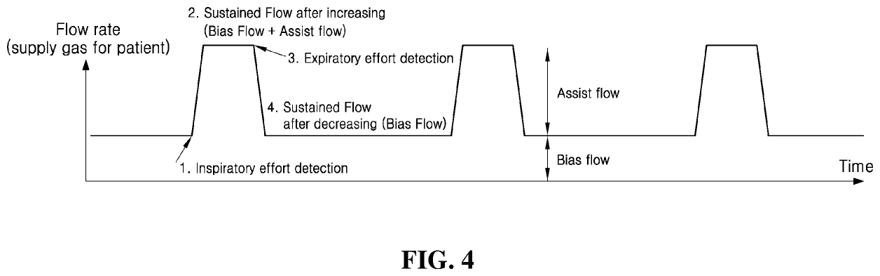

For the sake of convenience in understanding, FIG. 3 will be described with reference to the waveforms illustrated in FIGS. 4 to 6.

The control part 150A of the high flow respiratory therapy device according to the first embodiment of the present invention includes: a breathing synchronization part 1510, which includes a breathing pattern monitoring part 1511, an inspiratory effort point detection part 1512, and an expiratory effort point detection part 1513; and a supply flow control part 1520 according to inhalation/exhalation.

Here, the supply flow control part 1520 according to inhalation/exhalation is configured to control a supply flow rate in conjunction with the breathing synchronization part 1510, and even if it is a control part which controls the flow rate with the same function, may be distinguished according to the functional role.

The breathing pattern monitoring part 1511 monitors the flow rate change and the pressure change of the mixed gas supplied to the patient using the flow sensor and pressure sensor of the high flow respiratory therapy device 100 (in FIGS. 1 and 2). Through such monitoring, the breathing pattern monitoring part collects the patient's breathing pattern information.

In addition, the breathing pattern information and the respiratory effort information of FIG. 5 may include flow rate and pressure information detected from the flow sensor and the pressure sensor. The patient respiration-related waveform illustrated in FIG. 5 shows that the patient's flow rate increases at the inspiratory effort point and decreases at the expiratory effort point.

The inspiratory effort point detection part 1512 detects the inspiratory effort point A (in FIG. 5), at which the patient tries to start inhaling, from the patient's breathing pattern information collected in the breathing pattern monitoring part 1511.

Simultaneously, the expiratory effort point detection part 1513 detects the expiratory effort point B (in FIG. 5), at which the patient tries to start exhaling, from the breathing pattern information collected in the breathing pattern monitoring part 1511.

Specifically, the inspiratory effort point detection part 1512 extracts a maximum inspiratory effort point C in (FIG. 5), at which the change in inhalation flow rate is maximized, and an expiratory effort end point E (in FIG. 5), at which respiration temporarily stops before inhaling again after exhaling, from the patient's breathing pattern information. Thereafter, the inspiratory effort point detection part detects an inspiratory effort point A (in FIG. 5) at which the patient starts (initiates) an inspiratory effort in between the extracted expiratory effort end point E (in FIG. 5) and the maximum inspiratory effort point C (in FIG. 5), with reference to the extracted expiratory effort end point E and the maximum inspiratory effort point C.

At this time, the inspiratory effort point detection part 1512 can detect the expiratory effort end point from an average value obtained by adding the patient's respiratory flow rate during one cycle and dividing by the number of counts, and extract where the maximum inspiratory effort point and the maximum expiratory effort point are located relative to the expiratory effort end point as the zero point.

In addition, the inspiratory effort point detection part 1512 can calculate a scalar value between the expiratory effort end point E (in FIG. 5) in the first cycle and the maximum inspiratory effort point C (in FIG. 5) in the second cycle, referring to at least two cycles, and detect a value within a certain range of 10% to 30% from the maximum inspiratory effort point C (in FIG. 5) based on the calculated scalar value as the inspiratory effort point A (in FIG. 5). Alternatively, the inspiratory effort point detection part 1512 can calculate a gradient between the expiratory effort end point E (in FIG. 5) in the first cycle and the maximum inspiratory effort point C (in FIG. 5) in the second cycle, and detect the inspiratory effort point A (in FIG. 5) based on the calculated gradient.

The range of 10% to 30% can be set differently according to the patient's condition, the mounted state of the cannula, and so on.

The effort points and the end points are calculated in relation to the following equation.

Effort=Pressure×Volume/time=Pressure×Flow (L/min)

The expiratory effort point detection part 1513 extracts the maximum expiratory effort point D (in FIG. 5), at which the change in the patient's exhalation flow rate is maximized, and an expiratory effort endpoint E (in FIG. 5), at which respiration temporarily stops before inhaling again after exhaling, from the patient's breathing pattern information. Thereafter, the expiratory effort point detection part detects an expiratory effort point B (in FIG. 5) at which the patient starts (initiates) an expiratory effort in between the extracted expiratory effort end point E (in FIG. 5) and the maximum expiratory effort point D (in FIG. 5), with reference to the extracted expiratory effort end point E and the maximum expiratory effort point D.

The expiratory effort end point E (in FIG. 5) can be extracted from an average value obtained by adding the patient's respiratory flow rate during one cycle and dividing by the number of counts.

The expiratory effort point detection part 1513 can extract where the maximum expiratory effort point and the maximum expiratory effort point are located relative to the expiratory effort end point E (in FIG. 5) as the zero point.

Furthermore, the expiratory effort point detection part 1513 can calculate a scalar value between the expiratory effort end point E (in FIG. 5) in the first cycle and the maximum expiratory effort point D (in FIG. 5) in the second cycle, referring to at least two cycles, and detect a value within a certain range of 10% to 30% from the maximum expiratory effort point D (in FIG. 5) based on the calculated scalar value as the expiratory effort point. Alternatively, the expiratory effort point detection part can calculate a gradient between the expiratory effort end point E (in FIG. 5) in the first cycle and the maximum expiratory effort point D (in FIG. 5) in the second cycle, and detect the expiratory effort point based on the calculated gradient.

The detected inspiratory effort point A and expiratory effort point B correspond to the time points before becoming the maximum inspiratory effort point C and the maximum expiratory effort point D respectively. Therefore, when the flow rates at the time points of the inspiratory effort point and the expiratory effort point are controlled and supplied, the patient's inspiratory effort or expiratory effort is effectively reduced at the time point when the patient inhales or exhales to the maximum.

The respiration synchronization part 1510 is configured to include the breathing pattern monitoring part 1511, the inspiratory effort point detection part 1512, and the expiratory effort point detection part 1513, and synchronizes the inspiratory effort point and the expiratory effort point extracted from the inspiratory effort point detection part 1512 and expiratory effort point detection part 1513 in the patient's breathing pattern information. Accordingly, the respiration synchronization part 1510 synchronizes the inhalation point and the exhalation point in the patient's respiratory cycle.

The supply flow rate control part 1520 synchronizes and performs the flow rate control of the mixed gas with respect to the inspiratory effort point and the expiratory effort point synchronized in the respiration synchronization part 1510.

That is, in the patient's breathing cycle, when reaching at the inspiratory effort point, the supply flow control part 1520 synchronizes at the point and increases the flow rate of the mixed gas, and when reaching the expiratory effort point, the supply flow control part decreases the flow rate of the mixed gas.

At this time, the supply flow control part 1520 sets a flow rate that can ventilate the patient's nasal cavity with fresh air as a basic flow (bias flow). The supply flow control part 1520 usually supplies air at the bias flow, but supplies air by adding an plus auxiliary flow (assist flow) to the bias flow at the time of inhalation.

The assist flow may vary according to the patient's condition, the patient's breath size, and the mounted state of the cannula.

The flow rate to fill the nasal cavity with fresh air can be determined based on clinical or anatomical criteria.

Therefore, in a case in which the patient's breathing starts or the operation of the cannula starts after the cannula is installed, the high flow respiratory therapy device according to the first embodiment of the present invention supplies the mixed gas of the bias flow through the control of the control part 150A, and supplies the inhalation flow rate which increased by the assist flow amount from the bias flow when reaching the inspiratory effort point. when the patient's inhalation ends and the expiratory effort point is detected after continuously supplying the inhalation flow rate, the high flow respiratory therapy device performs an operation to reduce the inhalation flow rate by switching to the bias flow.

Through the above operation, the supply flow rate like the waveform illustrated in FIG. 4 is obtained.

Meanwhile, the waveform illustrated in the lower portion of FIG. 5 is the waveform of the patient's breathing effort as a result of performing the respiration synchronization process based on the bias flow in the device according to the first embodiment of the present invention.

The baseline where the cycle starts in FIG. 5 corresponds to a value corresponding to the bias flow.

On the other hand, FIG. 6 illustrates the waveform of the patient's breathing effort as a result of performing the breathing synchronization process not only based on the bias flow but also the inhalation flow in the device according to the first embodiment of the present invention.

That is, the first half of the cycle is a waveform performed by the breathing synchronization process at the bias flow, and the latter half of the cycle is a waveform performed by the breathing synchronization process at the inhalation flow (bias flow+assist flow). It is confirmed that the baseline between the first half of the cycle and the latter half of the cycle has changed.

Such a synchronization method can be applied when the therapeutic flow rate changes, and may be a process of breathing synchronization to match a new flow rate.

Meanwhile, in the embodiment of FIG. 6, when detecting the inspiratory effort point and the expiratory effort point, with respect to the mixed gas flow rate of FIG. 4, the inspiratory effort point occurs when supplying the bias flow, and the expiratory effort point occurs when supplying the inhalation flow. Therefore, in a case of detecting each effort point, especially when detecting the inspiratory effort point, it is advantageous to synchronize the bias flow and detect it at the bias flow, and when detecting the expiratory effort point, it is advantageous to detect it at the inhalation flow. This is one example of detection methods and is not limited thereto, and it would be also possible to detect the effort points at the bias flow regardless of the flow rate.

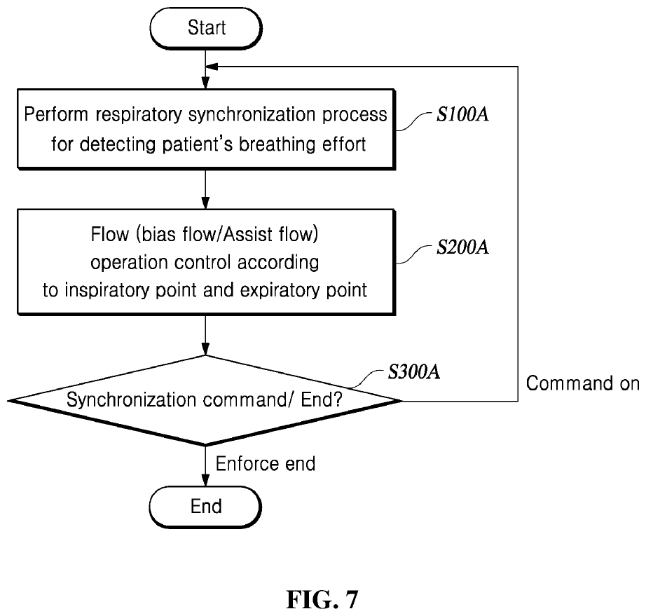

Meanwhile, FIG. 7 is an operation flow chart for depicting a high flow respiratory therapy method through breathing synchronization according to the first embodiment of the present invention.

In step S100A, the control part 150A performs a breathing synchronization process to detect the patient's respiratory effort.

Specifically, the breathing pattern monitoring part 1511 in the control part 150A monitors changes in flow rate and pressure of the mixed gas supplied to the patient and collects information on the patient's breathing pattern.

Subsequently, the detection part of the control part 150A detects an inspiratory effort point at which the patient is trying to start inhaling, and an expiratory effort point at which the patient is trying to start exhaling, or only the expiratory effort point, from the patient's breathing pattern information collected in the previous process.

For example, the inspiratory effort point detection part 1512 of the control part 150A detects the inspiratory effort point, and the expiratory effort point detection part 1513 of the control part 150A detects the expiratory effort point.

Thereafter, the breathing synchronization unit 1510 of the control part 150A synchronizes the detected inspiratory effort point and expiratory effort point with the patient's breathing pattern information.

Generally, the start of the patient's inhalation depends on the patient's will to breathe. Through the breathing synchronization process, the high flow respiratory therapy device can synchronize the actual inhalation point and the actual exhalation point of the patient with the respiratory cycle, respectively.

In the step S200A, the supply flow control part 1520 of the control part 150A actively controls the flow rate of the mixed gas corresponding to the inspiratory effort point or the expiratory effort point, and especially, performs flow control in synchronization with the inspiratory effort point and the expiratory effort point, which are synchronized in the breathing synchronization unit.

Specifically, the supply flow control part 1520 supplies mixed gas of a predetermined bias flow when the patient's respiration starts, supplies the mixed gas by adding assist flow to the bias flow at the inspiratory effort point, and supplies the mixed gas by reducing to the bias flow at the expiratory effort point.

In the step S300A, if there is an additional command for synchronization by the manipulation of the user or the doctor, the control part 150A moves back to the step S100A and operates, and stops the operation when synchronization end is input.

### Second Embodiment

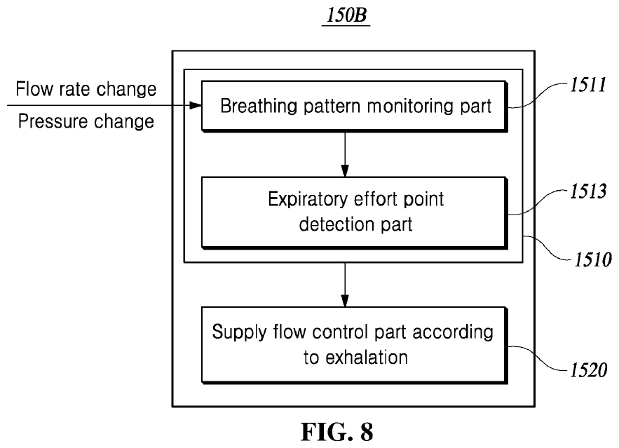

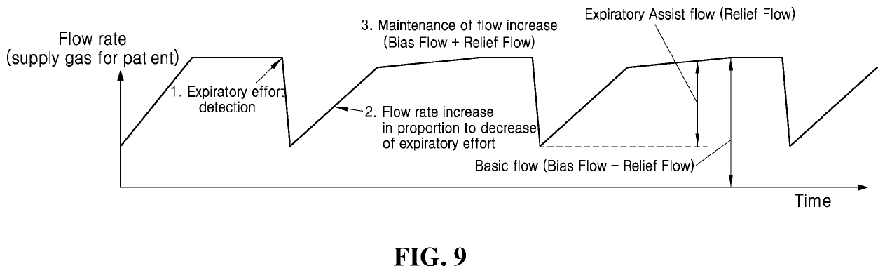

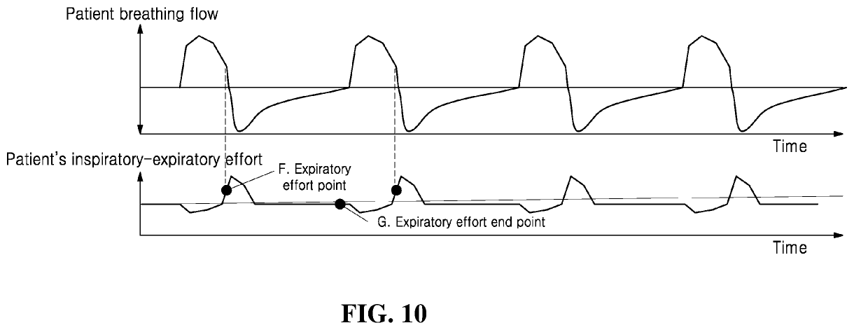

FIG. 8 is a diagram illustrating the configuration of a high flow respiratory therapy device according to a second embodiment of the present invention, FIG. 9 is a graph showing a value that changes in flow rate of a mixed gas are monitored according to the second embodiment of the present invention, and FIG. 10 is a waveform diagram illustrating an example of extracting an inspiratory effort point and an expiratory effort point of a patient according to the patient's respiratory changes according to the second embodiment of the present invention.

Referring to FIG. 8, the high flow respiratory treatment device according to the second embodiment of the present invention includes a control part 150B including a breathing synchronization unit 1510, which has a breathing pattern monitoring part 1511, and an expiratory effort point detection part 1513, and a supply flow control part 1520.

Each unit performs the same functions as the components according to the first embodiment. However, in the second embodiment, the supply flow control part 1520 of the second embodiment is different from that of the first embodiment in that the supply flow control part 1520 of the second embodiment detects only the expiratory effort point to control the flow rate of the mixed gas.

The breathing pattern monitoring part 1511 monitors at least one of the flow rate change and the pressure change of the mixed gas supplied to the patient, and collects information on the patient's breathing pattern.

The expiratory effort point detection part 1513 detects an expiratory effort point, at which the patient tries to start exhalation, from the breathing pattern information collected by the breathing pattern monitoring part 1511. The detection method is the same as described above.

The breathing synchronization unit 1510 includes the breathing pattern monitoring part 1511 and the expiratory effort point detection part 1513, and synchronizes an expiratory effort point from the expiratory effort point detection part 1513 with the patient's breathing pattern information. Through this, an exhalation point is synchronized with the patient's respiratory cycle.

The supply flow control part 1520 also synchronizes the control of the mixed gas flow with respect to the expiratory effort point synchronized in the breathing synchronization unit 1510.

In other words, when the patient's respiration starts, the supply flow control part 1520 supplies and maintains air at a predetermined bias flow, and reduces the flow rate of the mixed gas by an exhalation minus-auxiliary flow (relief flow) at an expiratory effort point and supplies the exhalation flow, thereby reducing the patient's expiratory effort. Thereafter, when the expiratory effort is reduced, as illustrated in FIG. 9, the supply flow control part 1520 increases the flow rate in proportion to the decrease of the expiratory effort. When the flow rate is increased to the bias flow, the supply flow control part 1520 maintains the bias flow till the next expiratory effort point is detected.

In the second embodiment, the bias flow is determined to include the flow level which can ventilate the patient's nasal cavity with fresh air and the patient's maximum inhalation flow.

The patient's maximum inhalation flow can be determined to be three to four times the breathing capacity per minute clinically. The breathing capacity per minute is calculated as [one breath volume]×[number of breaths per minute].

For example, assuming that one breathing capacity for adults is 500 ml and the average number of breaths per minute is 14, the breathing capacity per minute is approximately 7 L/min, and accordingly, the maximum inhalation flow is determined to be 20 to 30 LPM.

As described above, the flow level to fill the nasal cavity with fresh air and the patient's maximum inhalation flow can be determined based on clinical or anatomical criteria.

FIG. 10 shows the waveform for the patient's expiratory effort as a result of performing the breathing synchronization process of the device according to the second embodiment based on the bias flow.

Hereinafter, the operation of the high flow respiratory treatment device according to a second embodiment of the present invention will now be described.

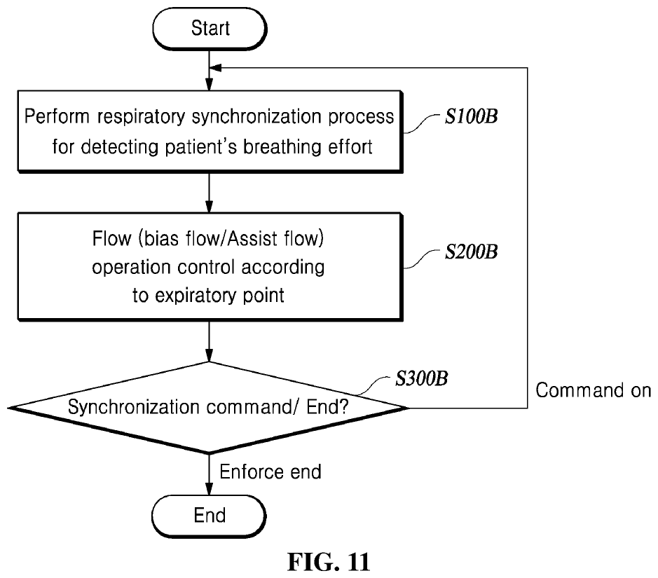

Referring to FIG. 11, in step S100B, the control part 150B performs a breathing synchronization process to detect the patient's respiratory effort.

Specifically, the breathing pattern monitoring part 1511 in the control part 150B monitors the flow and pressure changes of the mixed gas supplied to the patient, and collects information on the patient's breathing pattern.

Subsequently, the expiratory effort point detection part 1513 in the control part 150B detects only the expiratory effort points at which the patient tries to start exhalation from the collected patient's breathing pattern information through the above process.

Thereafter, the breathing synchronization unit 1510 in the control part 150B synchronizes the detected expiratory effort points with the patient's breathing pattern information.

In the next step S200B, the supply flow control part 1520 in the control part 150B actively controls the flow rate of the mixed gas corresponding to the expiratory effort point, in particular, can synchronize the flow control with the expiratory effort point synchronized in the breathing synchronization unit 1510.

Specifically, the supply flow control part 1520 supplies mixed gas of a predetermined bias flow when the patient's respiration starts, and then, and reduces the flow rate of the mixed gas by an exhalation minus auxiliary flow (relief flow) at an expiratory effort point and supplies the exhalation flow. Thereafter, the supply flow control part 1520 increases the flow rate in proportion to the decrease of the expiratory effort, and when reaching the bias flow, maintains the bias flow till the next expiratory effort is detected.

In step S300B, if there is an additional command for synchronization by the manipulation of the user or the doctor, the control part 150B moves back to the step S100B and operates, and stops the operation when synchronization end is input.

The above description merely illustrates the invention as an example, and various modifications are possible within the scope not departing from the technical idea of the invention by a person having ordinary knowledge in the technical field to which the invention belongs. Therefore, the embodiments disclosed in the specification of the invention do not limit the present invention. The scope of the invention should be interpreted by the following claims, and various equivalents and modification examples that can replace them at the time of this application should be interpreted as included in the scope of the invention.

### MODE FOR INVENTION

Various embodiments have been described in the best form to carry out the present invention.

### INDUSTRIAL APPLICABILITY

The invention is used in fields related to the high flow respiratory therapy device and method through breath synchronization.

It is obvious to those skilled in the art that various changes and modifications can be made in the invention without departing from the concept or scope of the invention. Therefore, it is intended that the present invention includes changes and modifications provided within the scope of the attached claims and their equivalents.

1. A high flow respiratory therapy device through breath synchronization comprising:
a breathing pattern monitoring part, which monitors flow rate and pressure changes of a mixed gas supplied to a patient and collects the patient's breathing pattern information;
an inspiratory effort point detection part, which detects an inspiratory effort point, at which the patient tries to start inhalation, from the patient's breathing pattern information;
an expiratory effort point detection part, which detects a expiratory effort point, at which the patient tries to start exhalation, from the patient's breathing pattern information; and
a supply flow control part, which increases the flow of the mixed gas when the patient's respiration becomes the inspiratory effort point and decreases the flow of the mixed gas when the patient's respiration becomes the expiratory effort point.

2. The high flow respiratory therapy device according to claim 1, further comprising:
a breathing synchronization unit, which includes a breathing pattern monitoring part, an inspiratory effort point detection part, and an expiratory effort point detection part, and synchronizes the extracted inspiratory effort point and the expiratory effort point with the patient's breathing pattern information,
wherein the supply flow control part synchronizes the flow control of the mixed gas with respect to the inspiratory effort point and expiratory effort point synchronized in the breathing synchronization unit.

3. The high flow respiratory therapy device according to claim 1, wherein the supply flow control part sets a basic flow (bias flow), which includes a flow level capable of ventilating the patient's nasal cavity with fresh air and the patient's maximum inhalation flow, and supplies the mixed gas of the bias flow when the patient's respiration starts, increases and supplies the flow rate by adding an plus auxiliary flow (assist flow) to the bias flow at the inspiratory effort point, and reduces the flow rate to the bias flow at the expiratory effort point.

4. The high flow respiratory therapy device according to claim 3, wherein the patient's maximum inhalation flow is calculated to be three to four times the patient's respiration rate per minute.

5. The high flow respiratory therapy device according to claim 1, wherein the inspiratory effort point detection part extracts a maximum inspiratory effort point, at which the change in inhalation flow rate is maximized, and an expiratory effort end point, at which respiration temporarily stops before inhaling again after exhaling, from the patient's breathing pattern information, and detects an inspiratory effort point at which the patient starts an inspiratory effort between the extracted expiratory effort end point and the maximum inspiratory effort point referring to the extracted expiratory effort endpoint and the maximum inspiratory effort point.

6. The high flow respiratory therapy device according to claim 1, wherein the expiratory effort point detection part extracts a maximum expiratory effort point, at which the change in the patient's exhalation flow rate is maximized, and an expiratory effort end point, at which respiration temporarily stops before inhaling again after exhaling, from the patient's breathing pattern information, and detects an inspiratory effort point at which the patient starts an inspiratory effort between the extracted maximum expiratory effort point and the expiratory effort end point referring to the extracted maximum expiratory effort point and the expiratory effort end point.

7. A high flow respiratory therapy device through breathing synchronization comprising:
a breathing pattern monitoring part, which monitors one or more of flow and pressure changes of a mixed gas supplied to a patient and collects patient's breathing pattern information;
an expiratory effort point detection part, which detects an expiratory effort point at which the patient tries to start exhalation from the collected breathing pattern information; and
a supply flow control part, which supplies the mixed gas of a set basic flow when the patient's respiration starts, and reduces the flow rate of the mixed gas by an minus auxiliary exhalation flow rate (relief flow) at the expiratory effort point to reduce the patient's expiratory effort.

8. The high flow respiratory therapy device according to claim 7, further comprising:
a breathing synchronization unit, which includes a breathing pattern monitoring part, and an expiratory effort point detection part and synchronizes the extracted expiratory effort point with the patient's breathing pattern information, and
wherein supply flow control part synchronizes the flow control of the mixed gas according to the expiratory effort point synchronized in the breathing synchronization unit.

9. The high flow respiratory therapy device according to claim 7, wherein the supply flow control part is set to include a flow level capable of ventilating the patient's nasal cavity with fresh air and the patient's maximum inhalation flow, and
wherein the patient's maximum inhalation flow is calculated to be three to four times the patient's respiration rate per minute.

10. The high flow respiratory therapy device according to claim 7, wherein the expiratory effort point detection part extracts a maximum expiratory effort point, at which the change in the patient's exhalation flow rate is maximized, and an expiratory effort end point, at which respiration temporarily stops before inhaling again after exhaling, from the patient's breathing pattern information, and detects an inspiratory effort point at which the patient starts an inspiratory effort between the extracted maximum expiratory effort point and the expiratory effort end point referring to the extracted maximum expiratory effort point and the expiratory effort end point.

11. A therapy method in a high flow respiratory therapy device, comprising:
a step in which a breathing pattern monitoring part monitors flow rate and pressure changes of a mixed gas supplied to a patient and collects the patient's breathing pattern information;
a step in which a detection part of a control part detects an inspiratory effort point, at which the patient tries to start inhalation, and an expiratory effort point, at which the patient tries to start exhalation, from the patient's breathing pattern information, or detects only the expiratory effort point; and
a step in which a supply flow control part of the control part actively controls the flow rate of the mixed gas in response to the inspiratory effort point or the expiratory effort point.

12. The therapy method according to claim 11, further comprising: after the step of detecting,
a step in which the breathing synchronization part of the control part synchronizes the detected inspiratory effort point or the expiratory effort point with the patient's breathing pattern information, and
wherein in the step of controlling the flow of the mixed gas, the supply flow control part of the control part synchronizes the flow control of the mixed gas in response to the inspiratory effort point and expiratory effort point synchronized in the breathing synchronization part.

13. The therapy method according to claim 11, wherein in the step of detecting, in a case in which the inspiratory effort point and the expiratory effort point are detected,
in the step of controlling the flow of the mixed gas, the supply flow control part supplies the mixed gas of the bias flow when the patient's respiration starts, increases and supplies the flow rate by adding an plus auxiliary flow (assist flow) to the bias flow at the inspiratory effort point, and reduces and supplies the flow rate to the bias flow at the expiratory effort point.

14. The therapy method according to claim 11, wherein in a case in which only the expiratory effort point is detected in the step of detecting,
in the step of controlling the flow rate of the mixed gas, the supply flow control part supplies the mixed gas of a set basic flow when the patient's respiration starts, and reduces the flow rate of the mixed gas by an minus auxiliary exhalation flow rate (relief flow) at the expiratory effort point, and then, increases the flow rate to the basic flow in proportion to the decrease in expiratory effort, thereby reducing the patient's expiratory effort.
